(19) United States
(12) Patent Application Publication
(10) Pub. No.: US 2005/0072432 A1
(43) Pub. Date: Apr. 7, 2005

(54) Urinary condom and method for its manufacture

(75) Inventor: Werner Kepp, Waiblingen (DE)

Correspondence Address: HOWSON AND HOWSON;ONE SPRING HOUSE CORPORATION CENTER, BOX 457, 321 NORRISTOWN ROAD, SPRING HOUSE, PA, 19477, US

(73) Assignee: Inmed Corporation, Duluth, GA (US)

(21) Appl. No.: 10/877,056

(22) Filed: Jun. 25, 2004

(30) Foreign Application Priority Data
Jun. 27, 2003 (DE) 103 29 129.6

(51) Int. Cl. A61F 6/04

(52) U.S. Cl. 128/844; 604/544

(57) ABSTRACT

A method is proposed for manufacturing a urinal condom with a reinforced tip and a sheath part, wherein the urinal condom has at least one polyurethane base layer, and in which to fabricate the base layer and/or reinforced tip, a dip molding sequence: Providing a dipping bath comprising an anionically stabilized aqueous dispersion of an aliphatic polyurethane, with an anionic stabilizer, preferably an anionic surfactant, being incorporated into the molecular chains of the polyurethane, so as to anionically stabilize the aqueous dispersion, Immersing a dipping form corresponding to the desired urinal shape into a dipping bath consisting of an aqueous dispersion of an aliphatic polyurethane, taking the dipping form out of the dipping bath, wherein a layer of the dispersion adheres to the dipping form, and creating a polyurethane layer by drying the dispersion adhering to the dipping form, is executed at least once, preferably twice for making the sheath part.

The invention relates to a latex-free urinary condom for male patients, in particular an external catheter for collecting and draining involuntary urinary discharges, and a method for manufacturing the urinary condom. Such urinary condoms have a condom-like sheath part and a reinforced tip consisting of a cylindrical catheter part and a conical intermediate piece for joining the tip and sheath part.

Patent DE 3236396C2 discloses a urinary condom, but one which is not latex-free. The sheath part of the urinary condom is comprised of a latex layer, an adhesive layer and a separating layer made of silicone rubber between the latex layer and adhesive layer. The sheath part is rolled up toward the tip in such a way as to roll in the adhesive layer between the separating layer and base layer, wherein the adhesive layer adheres more strongly to the base layer than to the separating layer. When the rolled-up sheath part of the urinary condom is unrolled over the penis of the urinary condom user, the adhesive layer adheres to the base layer, as a result of which the base layer sticks to the penile skin. The disadvantage to the urinary condom is that latex is a material to which many people have an allergic reaction. In addition, latex has low moisture permeability, and the sheath part of urinary condoms made of latex has to be made with relatively thick walls, further reducing the moisture permeability of the urinary condom. During long-term use, this low moisture permeability leads to skin irritations, inflammations and similar skin conditions. Further, latex urinary condoms are opaque due to their thick walls, so that said conditions cannot be diagnosed early on.

Laid-Open Patent Specification WO 96/29963 discloses a urinary condom with a sheath part made of a polyurethane layer and its manufacture out of polyurethane in a liquid state. This liquid-state polyurethane is the prepolymer of polyurethane, which is cross-linked to polyurethane by heating. To improve the processing characteristics, use is made of processing additives, i.e., chemicals such as emulsifiers, thickeners and wetting agents, which are also present in the urinary condom and can lead to intolerance reactions. The urinary condom is manufactured in a dip molding process. The dipping form is dipped into a prepolymer bath and removed again from the prepolymer bath at a defined speed. The dipped-on prepolymer layer is cross-linked to polyurethane in 40 minutes while incrementally increasing the temperature from 60 to 190° C. After cooling to 60° C., the coating process is repeated until a desired layer thickness has been achieved. The reinforced tip is also fabricated in a dip molding process, but with a dipping bath mixture differing from that for the thin-walled sheath part. Therefore, the manufacturing process for the disclosed urinary condom is very complicated, and requires time and cost intensive cross-linking at high temperatures.

International Patent WO 96/08353 discloses a method for the manufacture of elastic articles, for example, condoms, which contain a polyurethane layer. The articles are manufactured from an anionically stabilized aqueous dispersion of a polyurethane by means of a dip molding process. The disadvantage of this method is that the dispersion contains additives, e.g., a softener and a surface-active substance, i.e., an anionic stabilizer. After production, these additives remain in the polyurethane layer. Since the additives are not stably incorporated into the molecular structure of the polyurethane layer, they can be released, e.g., evaporate, while the articles are in use, which can cause skin reactions. This disadvantage could be avoided by washing the finished article in a water bath. But this would entail considerably increased production costs.

The object of the invention is to provide a latex-free urinary condom and a method for its manufacture that avoids the disadvantages of prior art and, in particular, is easy, quick and inexpensive to fabricate and use while largely avoiding skin reactions.

This object is achieved by the method and device described in the independent claims. The dependent claims represent preferred embodiments of the invention.

According to the invention, the object is achieved by providing a urinary condom with a reinforced tip and sheath part, wherein the urinary condom has at least one polyurethane base layer, and the polyurethane layer is made of an aqueous dispersion of an aliphatic polyurethane which is anionically stabilized. According to this invention, an anionic stabilizer, preferably an anionic surfactant, is incorporated into the molecular chains of the polyurethane so as to anionically stabilize the aqueous dispersion. Aliphatic denotes an organic compound with open carbon chains in its structural formula. Such dispersions are anionically stabilized, so that no emulsifiers need be used. In contrast to the prior art, the dispersion therefore need not contain a freely dissolved anionic stabilizer. The polyurethane used is an aliphatic polyurethane, into the molecular chains of which an anionic stabilizer, preferably an anionic surfactant, is incorporated so as to anionically stabilize the aqueous dispersion, as a result of which the anionic groups, i.e., the stabilizer, are stably incorporated into the polyurethane polymer of the polyurethane layer of the urinary condom and cannot be released when the urinary condom according to the present invention is used. The reinforced tip here normally consists of a conical part that abuts the sheath part. The tapered end of the conical part is provided with a cylindrical catheter part, i.e., a flexible tube or hose section through which the urine can be drained. A significant advantage of the dispersion used according to the invention is that it contains no emulsifiers and no freely dissolved anionic stabilizers, so that the polyurethane layer of the urinary condom is also free of such chemical residues. As a result, the urinary condom according to the present invention is very well-tolerated by the skin. Processing aids or cross-linking agents need not be added. It is no longer necessary to wash the urinary condom during the production. Another favorable characteristic is that the polyurethane layer of the urinary condom according to the present invention can be readily coated. Once the coating is applied, it cannot react chemically with the released chemicals, in particular stabilizers, and/or be dissolved by the released chemicals or, in fact, even adhere to begin with.

Preferably, the dispersion and thus the polyurethane layer of the urinary condom are free from softeners. Polyurethanes containing softeners are especially unsuitable for use in the production of urinary condoms because they come in contact with the patients' skin for relatively long periods of time, thus making it possible for known incompatibilities of softeners to evolve to an especially great extent.

The polyurethane urinary condom according to the invention has very thin walls measuring at most 0.15 mm. By contrast, the wall thickness of urinary condoms made of other materials ranges from 0.25 to 0.3 mm. This permits a better adaptation to the skin, and allows for more reliable sealing in case of wrinkling. Further, the polyurethane urinary condom according to the invention has significantly higher water vapor permeability than urinary condoms made of silicone, latex or a thermoplastic elastomer (TPE). Polyurethane urinary condoms are transparent, and permit inspection of the skin while the urinary condom is being worn. The urinary condom according to the invention is manufactured by dipping in an emulsifier-free dispersion. Therefore, a prepolymer need not be cross-linked to polyurethane. Only drying takes place. Using an aqueous polyurethane dispersion while fabricating the urinary condom cuts down on the manufacturing time significantly. The urinary condom according to the invention can be made in a single dipping step. The number of dipping steps is not determined by the desired wall thickness of the urinary condom, but by other quality requirements, e.g., maximum tolerated rate of rejects.

The tip preferably has a reinforced layer, wherein the reinforced layer is fabricated from the aqueous dispersion of the aliphatic polyurethane using a coagulant, i.e., an agent that causes the polyurethane to flocculate out of the dispersion, preferably a calcium nitrate solution. The reinforced layer can both be applied to the base layer and abut the base layer of the sheath part. The advantage to manufacturing the reinforced tip with a coagulant is that, when applying the dispersion to form the tip, a thicker dispersion layer compared to non-use of a coagulant can be applied in one operation. This significantly cuts the time required to manufacture the urinary condom.

The sheath part of the urinary condom according to the invention preferably has a wall thickness of 0.05 to 0.1 mm. The tip preferably has a wall thickness of 0.1 to 0.5 mm. These wall thicknesses advantageously combine good water vapor permeability with a sufficient mechanical stability of the urinary condom.

In a preferred rolled-up embodiment, the sheath part of the urinary condom has at least one adhesive-repellant (anti-adhesive) separating layer and an adhesive layer, preferably made of acrylate adhesive. The separating layer is situated between the base layer and adhesive layer. The sheath part of the urinary condom is rolled up toward the tip in such a way as to roll in the adhesive layer between the separating layer and base layer, wherein the adhesive layer adheres more strongly to the base layer than to the separating layer. The advantage to this embodiment is that the adhesive adheres to the inside of the polyurethane base layer after unrolling the urinary condom during its use, thereby ensuring that the base layer of the urinary condom bonds with the skin of the penis onto which the urinary condom is unrolled. As a result, the urinary condom is easy to use and can be reliably applied.

The separating layer of the urinary condom preferably consists of a silicone rubber, and again preferably a silicone rubber with a cross-linking temperature of between 50 and 100° C. The advantage here is that silicone has good anti-adhesive properties, and the base layer need not be completely dried at the low cross-linking temperatures.

In terms of the method, the object is achieved through the use of a dip molding process to fabricate the urinary condom when forming the base layer and/or reinforced tip. The dip molding sequence is as follows:
(a) Provide a dipping bath comprising an aqueous dispersion of an aliphatic polyurethane which is anionically stabilized, with an anionic stabilizer, preferably an anionic surfactant, being incorporated into the molecular chains of the polyurethane so as to anionically stabilize the aqueous dispersion.
(b) Immerse a dipping form corresponding to the desired urinal shape into a dipping bath consisting of an aqueous dispersion of an aliphatic polyurethane;
(c) Take the dipping form out of the dipping bath, wherein a layer of the dispersion adheres to the dipping form, and
(d) Create a polyurethane layer by drying the dispersion adhering to the dipping form.

The dip molding sequence is executed at least once, preferably twice when forming the sheath part.

The use of an aqueous dispersion according to the invention eliminates the need for cross-linking after the dip molding process, requiring only a drying step. This accelerates the procedure.

To manufacture the reinforced tip, a coagulant, preferably a calcium nitrate solution, is preferably applied to the dipping form in the area of the urinary condom tip before executing the dip molding sequence. The coagulant can thus be applied to the dipping form in the area of the tip immediately prior to an initial dip molding sequence, or the coagulant is applied to the base layer in the area of the urinary condom tip on the dipping form prior to an additional dip molding sequence. After the dipping form is taken out of the dipping bath, a reinforced dispersion layer generated by the coagulant adheres to the base layer or the dipping form. The reinforcing layer or reinforced tip is again formed by drying the adhering dispersion.

The coagulant is preferably applied by immersing the area of the urinary condom tip or dipping form tip into a dipping bath consisting of coagulant solution. The polyurethane dispersion is processed in a coagulant dip molding procedure, so that just one dipping step in the polyurethane dispersion is sufficient for the reinforced tip as well. According to known prior art, at least six to seven dipping steps are required for the reinforced tip.

After the dip molding sequence or sequences, a separating layer preferably made of silicone rubber is preferably applied to the polyurethane layer of the urinary condom sheath part and an adhesive layer to the separating layer, preferably via atomization. The separating layer is preferably applied to the dipping form by dipping the sheath part of the polyurethane layer on the dipping form into a silicone rubber bath or applying an atomized rubber layer to the sheath part of the polyurethane layer. A silicone rubber having a cross-linking temperature of between 50 and 100° C. is preferably used to create the separating layer. The advantage to atomized coating is that no complicated dip molding system need be provided for the silicone layer and/or the adhesive.

The urinary condom fabricated according to the invention is preferably removed from the dipping form by rolling up the sheath part of the urinary condom toward the tip. This yields the preferred rolled-up embodiment of the urinary condom during removal from the dipping form.

When using the preferred, rolled-up embodiment of the urinary condom, the rolled-up sheath part of the urinary condom is unrolled over the penis of the urinary condom user, wherein the adhesive layer adheres to the base layer, as a result of which the base layer sticks to the penile skin.

Additional advantages are described in the specification and attached drawings. The features of the invention mentioned above and further below can each be used separately or in combination. The mentioned embodiments are not to be regarded as a final tally, but rather are exemplary in nature.

The invention shall be explained in greater detail below based on exemplary embodiments, with reference to the drawings:

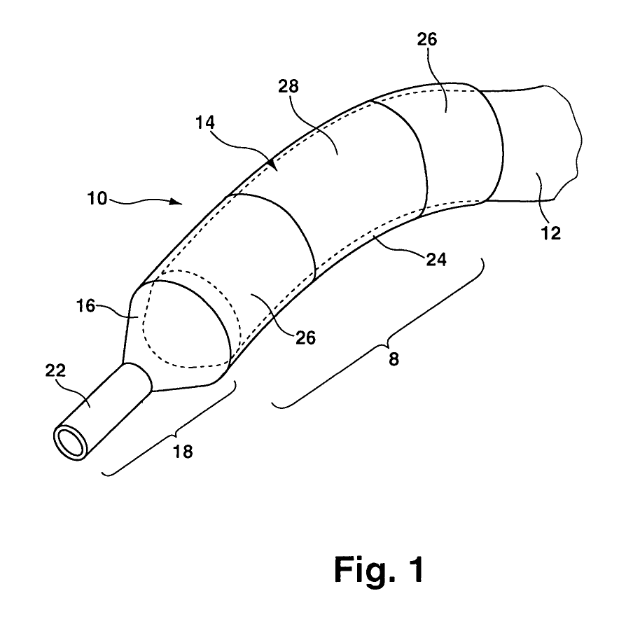

FIG. 1 shows a urinary condom according to the invention in use;

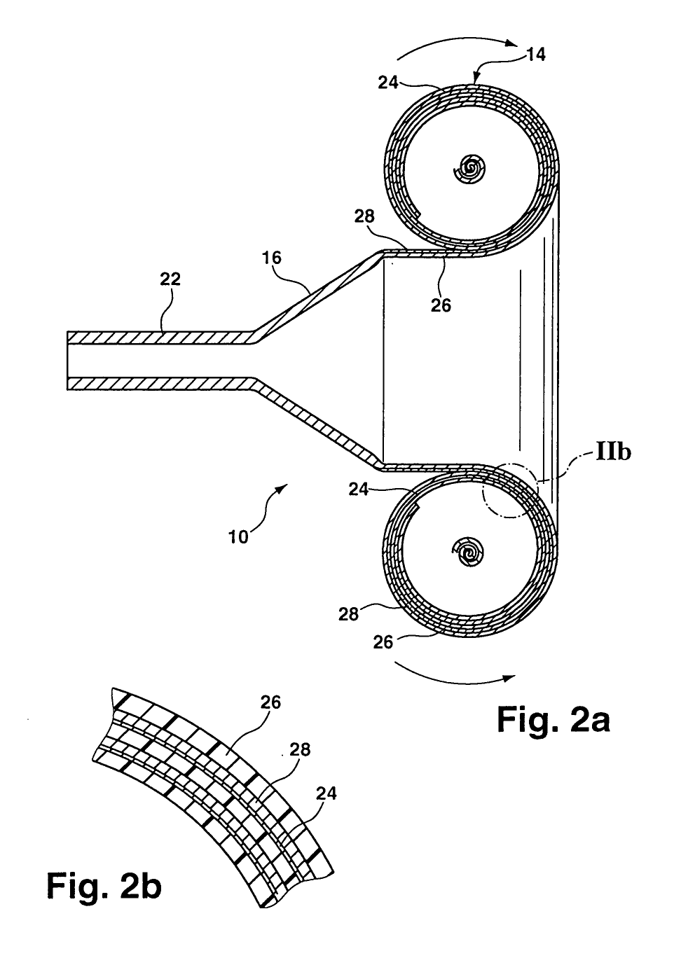

FIG. 2 (FIGS. 2a and 2b) shows a urinary condom according to the invention in a preferred embodiment, rolled up;

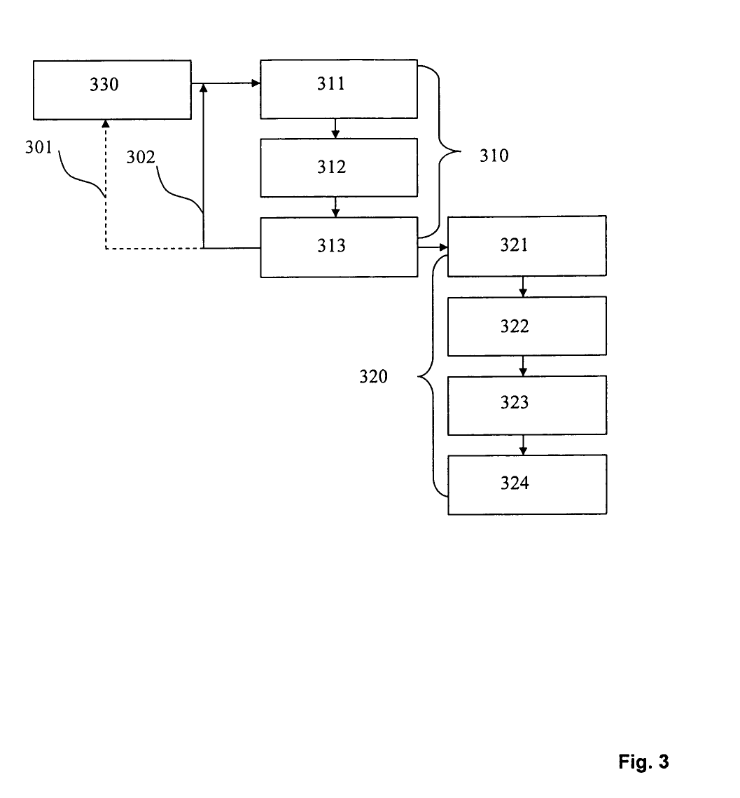

FIG. 3 shows a flow chart of the procedure for manufacturing a urinary condom according to the invention; and

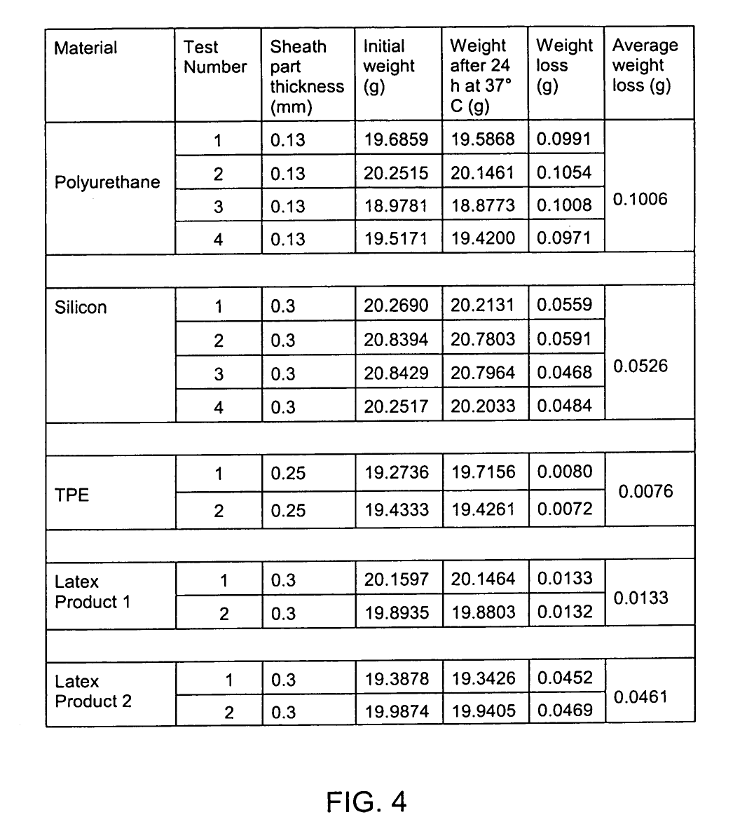

FIG. 4 shows a table relating to the water vapor permeability of urinary condoms made of various materials.

The drawings of the object according to the invention shown on the figures are highly schematic and not to scale. The individual constituents of the object according to the invention are depicted in such a way as to illustrate their structure.

In FIG. 1, the self-adhesive urinary condom 10 according to the invention is shown in use, i.e., after the sheath part 8 has been unrolled over the penis 12 of the urinary condom user. The layer sequence of the sheath part 8 can be discerned from the figure. The reinforced tip 18 of the urinary condom 10 consists of a tube 22 and a conical part 16. As shown, the sheath part 8 is made up of the following layer sequence when applied to the penis 12: adhesive layer 24, polyurethane base layer 26 and separating layer 28. The polyurethane base layer is shown in the figure in a highly diagrammatic manner by two bordering lines in a midsection of the sheath part, uncovering the separating layer. The adhesive layer 24 was applied to the separating layer 28 while manufacturing the urinary condom 10. The adhesive layer 24 is only introduced between the penile skin and base layer 26 during use while in the process of unrolling, since the adhesive adheres more strongly to the inside of the base layer 14 than to the separating layer 28. The outside of self-adhesive urinary condoms must be adhesive-repellent (anti-adhesive), so that the rolled-up urinary condom can be unrolled again. This is achieved via the anti-adhesive separating layer applied to the polyurethane base layer 26. The separating layer 28 consists of a silicone rubber. Preferably involved here is a silicone rubber that cross-links within a few seconds at temperatures clearly below 100° C., e.g., in 4.6 s at 80° C. This low cross-linking temperature and the short cross-linking time are very advantageous for an efficient manufacturing process. Silicone that cross-links at room temperature can basically be used. However, this leads to long cross-linking times. The disadvantage to using silicone rubber that cross-links at over 100° C. is that the polyurethane layer must be completely dried before it is applied, resulting in very long drying periods.

FIGS. 2a and 2b in FIG. 2 depict the preferred, rolled-up embodiment of the self-adhesive urinary condom according to the invention. The sheath part of the urinary condom consists of a polyurethane base layer 26 made from an aqueous dispersion of an aliphatic polyurethane, an adhesive-repellant separating layer 28 and an adhesive layer 24. The adhesive-repellant separating layer is made of a silicone that guarantees rapid cross-linking at a temperature of below 100° C. The base layer 26 has an inner surface 14 and an outer surface. The adhesive-repellant separating layer 28 is applied to the outer surface of the base layer 26. In addition, the adhesive-repellant layer 28 has applied to it an adhesive layer 24 covered by the inner surface 14 of the base layer 26 with the urinary condom rolled up, so that the surfaces which are exposed when the urinary condom is rolled up have no adhesive properties. When the urinary condom is unrolled, the inner surface 14 of the base layer 26 picks up the adhesive layer 24 lying on the adhesive repellant separating layer 28, because it adheres more strongly to the adhesive layer 24, and takes this adhesive layer 24 along completely, so that the adhesive layer 24 is now on the inner surface 14 of the base layer 26 in the completely unrolled area of the urinary condom. As a result, the inner surface 14 of the base layer 26 sticks to the penile skin with the urinary condom unrolled, i.e., in use. The outside of self-adhesive urinary condoms must be adhesive-repellant (anti-adhesive), so that the rolled-up urinary condom can be unrolled again. This is accomplished with the anti-adhesive separating layer, which is applied to the polyurethane base layer. The separating layer 28 consists of a silicone rubber, preferably with a cross-linking temperature of under 100° C.

FIG. 2b shows a magnified view of the layer sequences on IIb from FIG. 2a.

FIG. 3 illustrates the manufacture of the urinary condom according to the invention using an aqueous dispersion of an aliphatic polyurethane in a flowchart. Manufacturing takes place via a dip molding process. In this case, use is made of a dip molding sequence 310 involving the steps of immersing a dipping form corresponding to the desired urinary condom shape into a dipping bath containing the dispersion 311, taking the dipping form out 312 of the dipping bath and forming the polyurethane layer via drying 313. The dipped-on layers (dipping layer) of the dispersion are hence only dried. This can basically take place at room temperature; drying at a temperature of 40 to 60° C. is preferred. These temperatures enable a sufficiently complete drying of the dipping film in less than six minutes.

The reinforced tip is manufactured using a coagulant, preferably a calcium nitrate solution. Prior to immersion in the polyurethane dispersion, the tip area of the dipping form is dipped in coagulant, i.e., a coagulant is applied in the area of the tip (reference number 330). During subsequent dipping in the polyurethane dispersion, the coagulant in the tip area causes a uniformly thick layer to become deposited on the dipping form. The layer thickness, which preferably measures at most 0.5 mm, is determined by the retention time of the dipping form in the dipping bath, and by the concentration of the used coagulant solution. A retention time of at most five minutes is preferred. Coagulant application 330 can take place either as a first manufacturing step before the dipping form is first immersed in the dispersion 311, or in an optional procedure 301, during repetition 302 of the dip molding sequence, between two dip molding sequences. The thin-walled, cylindrical sheath part of the urinary condom is not fabricated using a coagulant, but only via (preferably twofold) immersion in the dipping bath comprised of the polyurethane dispersion and drying of the dispersion adhering to the dipping form after the dipping form has been taken out of the dipping bath. The wall thickness of the sheath part preferably measures 0.07 to 0.09 mm, which ensures an optimum water vapor permeability and sufficient mechanical stability.

Separating layer application 321 takes place after drying the polyurethane base layer and reinforced tip. The outside of the cylindrical sheath part on the penis shaft side is coated with silicone, preferably by means of atomization (Coating). This is followed by separating layer cross-linking 322, i.e., the chemical hardening process for the silicone. After the silicone layer has been cross-linked, an adhesive layer, preferably acrylate adhesive, is applied 323 on top of the separating layer. This is also preferably accomplished through atomization. Finally, the sheath part of the urinary condom is rolled up 324 toward the tip, and removed from the dipping form in this way. The rolling-up step positions the adhesive layer between the inside and outside of the urinary condom. When unrolling the urinary condom for use, the adhesive layer adheres to the inside surface, or base layer, of the urinary condom, because the adhesive adheres more strongly to polyurethane than to silicone. The adhesive layer is then available for bonding the urinary condom to the skin of the penis shaft.

FIG. 4 shows a table relating to the water permeability of self-adhesive urinary condoms with a base layer comprised of different materials. The tested samples consist of three layers, specifically an adhesive layer, the base layer whose material was varied, and an adhesive-repellant separating layer. The silicone sample only has two layers, however, with no separating layer being present, since silicone has adhesive-repellant properties. To determine the measured values, 15 ml test tubes (inside diameter 15.5 mm) were filled with 14 ml of water and covered with the samples cut out of urinary condoms made of different materials, wherein the latter were sealed on the side with a clamping ring. The side of the urinary condoms coated with adhesive faced the water surface. The samples were taken from the center of the adhesive-coated sheath area of the urinary condoms. The test tubes sealed with the samples were weighed, and subsequently stored in a heating cabinet for 24 hours at 37° C., after which the sealed test tubes were again weighed. The weight loss determined in this way is a gauge for the water permeability of the sample materials. Among the urinary condoms tested in this way, the polyurethane urinary condom samples have the highest water vapor permeability. The silicone urinary condom samples only allow about half as much water vapor through under identical conditions. With respect to the latex material, products of two different manufacturers were tested. The two products vary greatly in water vapor permeability given the same wall thickness, measuring 45% of the polyurethane sample level in one case, and 13% on the other. A latex-free urinary condom made of TPE has the lowest permeability, measuring only about 8% of the polyurethane sample level. Water permeability is determined by both the material used for the sheath part and its wall thickness. The highly disparate results for the two latex urinary condoms show that, along with the base layer material, the separating layer and adhesive layer combine to determine the water vapor permeability.

The invention proposes a method for the production of a urinary condom with a reinforced tip and a sheath part, with the urinary condom having at least one base layer made of polyurethane, and with a dip molding sequence for the production of the base layer and/or the reinforced tip with the following steps: providing a dipping bath comprising an anionically stabilized aqueous dispersion of an aliphatic polyurethane with an anionic stabilizer, preferably an anionic surfactant, being incorporated into the molecular chains of the polyurethane so as to anionically stabilize the aqueous dispersion, immersing a dipping form corresponding to the desired shape of the urinary condom into a dipping bath, removing the dipping form from the dipping bath, with a layer of the dispersion adhering to the dipping form, and creating a polyurethane layer by drying the dispersion adhering to the dipping form, being carried out at least once and, to produce the sheath part, preferably twice.

### Reference List

8 Sheath part
10 Urinary condom
12 Penis
14 Base layer inside
16 Conical part
18 Reinforced tip
22 Tube
24 Adhesive layer
26 Polyurethane base layer
28 Separating layer
301 Optional procedure
302 Repetition of the dip molding sequence
310 Dip molding sequence
311 Immersion of dipping form into dispersion
312 Removal of dipping form
313 Forming of polyurethane layer via drying
320 Final manufacturing sequence
321 Separating layer application
322 Separating layer cross-linking
323 Adhesive layer application
324 Urinary condom roll-up
330 Coagulant application

1. A urinary condom with a reinforced tip and a sheath part, with the urinary condom having a least one base layer made of polyurethane and with the polyurethane layer being prepared from an anionically stabilized aqueous dispersion of a polyurethane, wherein the polyurethane is an aliphatic polyurethane into the molecular chains of which an anionic stabilizer is incorporated so as to anionically stabilize the aqueous dispersion.

2-14. (canceled)

15. The urinary condom according to claim 1, in which the anionic stabilizer is an anionic surfactant.

16. The urinary condom according to claim 1, in which the aqueous dispersion is free from softeners.

17. The urinary condom according to claim 1, in which the tip has a reinforced layer which is prepared from the aqueous dispersion of the aliphatic polyurethane using a coagulant.

18. The urinary condom according to claim 17, in which the coagulant is a calcium nitrate solution.

19. The urinary condom according to claim 1, in which the sheath part has a wall thickness of 0.05-0.1 mm

20. The urinary condom according to claim 1, in which the tip has a wall thickness of 0.1-0.5 mm.

21. The urinary condom according to claim 1, in which the sheath part has a wall thickness of 0.05-0.1 mm and the tip has a wall thickness of 0.1-0.5 mm.

22. The urinary condom according to claim 1, in which the sheath part has at least one adhesive-repellent separating layer and an adhesive layer, with the separating layer being disposed between the base layer and the adhesive layer, and with the sheath part being rolled up toward the tip in such a way that the adhesive layer comes to be rolled up between the separating layer and the base layer, with the adhesive layer adhering more strongly to the base layer than to the separating layer.

23. The urinary condom according to claim 22, in which the adhesive layer is made of acrylate adhesive,

24. The urinary condom according to claim 22, in which the separating layer consists of a silicone rubber.

25. The urinary condom according to claim 24, in which the silicone rubber has a cross-linking temperature of less than 100° C.

26. A method for the production of a urinary condom according to claim 1, in which at least one element from the group consisting of the base layer and the reinforced tip is produced by a dip molding process comprising the following steps:
(a) providing a dipping bath comprising an anionically stabilized aqueous dispersion of an aliphatic polyurethane, with an anionic stabilizer being incorporated into the molecular chains of the polyurethane so as to anionically stabilize the aqueous dispersion,
(b) immersing a dipping form corresponding to the desired shape of said element into the dipping bath,
(c) removing the dipping form from the dipping bath, with a layer of the dispersion adhering to the dipping form, and
(d) creating a polyurethane layer by drying the dispersion adhering to the dipping form.

27. The method according to claim 26, in which the anionic stabilizer is an anionic surfactant.

28. A method for the production of a urinary condom according to claim 1, in which the sheath part is produced by a dip molding process comprising the following steps:
(a) providing a dipping bath comprising an anionically stabilized aqueous dispersion of an aliphatic polyurethane, with an anionic stabilizer being incorporated into the molecular chains of the polyurethane so as to anionically stabilize the aqueous dispersion,
(b) immersing a dipping form corresponding to the desired shape of said sheath part into the dipping bath,
(c) removing the dipping form from the dipping bath, with a layer of the dispersion adhering to the dipping form, and
(d) creating a polyurethane layer by drying the dispersion adhering to the dipping form,
said steps being carried out at least twice.

29. The method according to claim 26, in which, to produce the reinforced tip, the processing step of applying a coagulant to the dipping form in the area of the tip of the urinary condom, is carried out prior to the dip molding process, the coagulant causing a reinforced dispersion layer to cling to the area of the tip.

30. The method according to claim 29, in which the coagulant is a calcium nitrate solution.

31. The method according to claim 29, in which the coagulant is applied to the dipping form in the area of the tip of the urinary condom by immersion in a dipping bath.

32. The method according to claim 26, in which, following the dip molding process, a separating layer is applied to the polyurethane layer of the sheath part of the urinary condom, and an adhesive layer is applied to the separating layer.

33. The method according to claim 32, in which, the separating layer is made of silicone rubber.

34. The method according to claim 32, in which the adhesive layer is applied by applying an atomizing coating.

35. The method according to claim 26, in which that the separating layer is applied by dipping the sheath part of the polyurethane layer on the dipping form into a silicone rubber bath, or by coating the sheath part of the polyurethane layer on the dipping form with an atomizing coating.

36. The method according claim 32, in which a silicone rubber with a cross-linking temperature of less than 100° C. is used as the separating layer.

37. A method for removing the urinary condom produced according to claim 32 from the dipping form, in which the sheath part of the urinary condom is removed from the dipping form by rolling the sheath part toward the tip.

38. A method for using the urinary condom according to claim 22, in which the rolled-up sheath part of the urinary condom is unrolled over the penis of the urinary condom user, with the adhesive layer remaining stuck to the base layer and with the base layer thus being made to adhere to the skin of the penis.
